(19) United States
(12) Patent Application Publication
(10) Pub. No.: US 2026/0196297 A1
(43) Pub. Date: Jul. 9, 2026

(54) SV DETECTION

(71) Applicant: SAGA Dx, Inc., Cambridge, MA (US)

(72) Inventors: Samuel Woodhouse, Lund (SE); Sergii Gladchuk, Lund (SE); Christopher Rushton, Lund (SE)

(21) Appl. No.: 19/442,701

(22) Filed: Jan. 7, 2026

Related U.S. Application Data
(60) Provisional application No. 63/743,149, filed on Jan. 8, 2025.

(51) Int. Cl. G16B 20/20; C12Q 1/6806; C12Q 1/6809; C12Q 1/686; C12Q 1/6869; G16B 25/20; G16B 30/20

(52) U.S. Cl. CPC G16B 20/20; C12Q 1/6806; C12Q 1/6809; C12Q 1/686; C12Q 1/6869; G16B 25/20; G16B 30/20

(57) ABSTRACT

Methods are provided for detecting SVs from sequencing data from a tumor sample and classifying those SVs as somatic or germline without the use of matched normal sequences. Methods use a database of SVs and SNP profiles from previous samples. Methods include comparing a subject SNP profile to sample SNP profiles in the database and excluding from further analysis database entries with matching SNP profiles (as presumably originating from the same subject). Where SVs from the tumor sample match SVs found in samples from other people, the tumor SVs are called as being germline. When the tumor SVs do not have any matches among data from other people, those SVs are called as somatic.

### BACKGROUND

Genetic variation involves both inherited germline variants and somatic mutations acquired after conception. Germline variants are typically found in all cells within a person while somatic variants are found in a limited subset of cells such as, for example, in the cells of a tumor. Whether germline or somatic, variants may involve polymorphisms or small insertions or deletions (“indels”) involving only a few bases. In other cases, variants are presents as large genomic rearrangements called structural variants (SVs), involving the deletion, duplication, or rearrangement of genomic segments greater than about 50 bases in length. SVs are infrequent compared to small polymorphisms, but often create loss- or gain-of-function mutations in cancer-related genes. SVs may be germline or somatic. While some somatic SVs are causative of cancer, others—both germline and somatic—may be benign, having minimal clinical significance.

Due to the contribution of somatic SVs to cancer, there has been interest in detecting somatic SVs. Established approaches to detecting tumor-specific SVs has involved analyzing genetic material from a tumor along with so-called “matched normal” samples. Using matched normal samples, the sequences from tumor DNA are compared to homologous sequences from non-tumor cells from the same person. However, matched normal samples are not always available. For background, see Yi, 2023, Toward the functional interpretation of somatic structural variations: bulk-and single-cell approaches, Briefings in Bioinformatics 24(5):bbad 297, incorporated by reference. Unfortunately, methods for detecting somatic SVs are unsatisfactory. For example, it is thought that attempts to detect tumor SVs without access to matched normal sequences is prone to false positives.

### SUMMARY

The invention provides methods for detecting SVs from sequencing data from a tumor sample and classifying those SVs as somatic or germline without the use of matched normal sequences. Methods of the invention make use of a database of genetic variants from previous samples. For each sample, the database includes SVs that were found in that sample as well as a record of single nucleotide polymorphisms, a sample SNP profile, associated with that sample. The tumor sample is from a subject and the database may include previous samples from that subject and from other people. To analyze the tumor sample, methods include using a subject SNP profile—a record of single nucleotide polymorphisms present in the subject—to compare to sample SNP profiles in the database. A sample in the database with a sample SNP profile that matches the subject SNP profile is presumed to be from the same person as the subject, albeit from previous visit or analysis. Database samples from the same person are excluded from further analysis, and the SVs from the tumor sample are compared to SVs in the non-excluded database records, which are from people other than the subject. Where SVs from the tumor sample match SVs found in samples from other people, the tumor SVs are called as being germline. When the tumor SVs do not have any matches among data from other people, those SVs are called as somatic.

It is extremely unlikely that a somatic SV in one tumor would have an identical match in another tumor. When a tumor SV matches an SV found in a sample from another subject, that SV is most likely a germline SV or an artifact introduced by sample preparation or bioinformatics. The SV may be provisionally “called” as germline or somatic, and once a tumor SV is provisionally called as somatic methods may include orthogonal validation, e.g., a secondary assay to confirm the detection of the somatic tumor SV. For examples, some bioinformatics pipelines for short reads from next-generation sequencing (NGS) instruments may mis-call an SV in certain genomic regions such as regions of gene duplication. In some embodiments, a provisional somatic SV call is confirmed by, for example, Sanger sequencing. That step of orthogonal validation may be used as evidence that the somatic SV is a candidate for use as a tumor biomarker.

Thus, methods of the invention involve the use of a database of SVs that have been previously identified for previous samples. The database can include previous samples from the same patient/subject, as well as from other people. Methods use a SNP profile as a personalized “fingerprint” to identify (and exclude) database records originating from the same person. The remaining database records are taken to come from other people, among which matching somatic SVs are extremely improbable. Thus, when the sample SV is found in the records from the other people, the SV is “called” as being germline. Those described methods are preferably implemented by a computer system.

Sequencing a tumor, e.g., using an NGS instrument, generates tumor sequence reads, typically in an electronic format such as a so-called FASTA or FASTQ file. The computer system can assemble those reads and/or map them to reference genomic data to detect SVs, and specifically to detect and report the genomic coordinates of the SVs and the breakpoints of those SVs. The initial SV detection may be performed by a software package such as the Perl/Cpp package for detection of structural variants from NGS reads known as BreakDancer. Methods make use of a subject SNP profile, which may be obtained from the sequence reads generated by sequencing the tumor, or which may be obtained by an independent assay such as a bead array.

The subject SNP profile is compared to sample SNP profiles in the database. When the subject and sample SNP profiles match, preferably according to pre-defined similarity criteria, the sample in the database is deemed to have come from the subject, e.g., at a prior clinical visit. The similarity criteria may be, for example, a requirement that some number of SNPs are found in both profiles or, e.g., a percent match. By matching SNP profiles, methods of the invention search for the SV among the results from other people. Finding the SV in samples from other people provide strong evidence that the SV is germline. Additionally, the described steps may be implemented entirely by software and may operate automatically when tumor DNA is being sequenced, e.g., by NGS. When a clinician initiates sequencing of tumor DNA, software modules may operate to automatically assemble and map FASTQ (or similar) reads from an NGS instrument, detect SVs from the tumor DNA and compare the associated SNP profile to the database and output the call as somatic or germline. That output gives the clinician actionable clinical information about the presence of somatic SVs in tumor DNA which may be used to guide treatment and patient care. For example, a somatic tumor SV may be used as a marked of minimal residual disease after treatment.

In certain aspects, the invention provides methods of detecting a tumor-specific mutation. Methods include identifying (i) a tumor structural variant (SV) in sequences of tumor DNA from a subject, and (ii) a subject SNP profile describing single nucleotide polymorphisms in nucleic acid of the subject. The subject SNP profile is compared to a database in which entries include sample SVs and a sample SNP profile from previous samples to select, from the database, a set of selected entries for which the sample SNP profile and the subject SNP profile are dissimilar according to a test. Methods further include searching the selected entries for the tumor SV and reporting the tumor SV as germline when the tumor SV matches at least one of the sample SVs in the selected entries. Methods may include reporting the tumor SV as somatic when the tumor SV does not find a match among the selected entries.

The entries in the database may be the result of analyzing the previous samples. The previous samples may include one or more samples from the subject and/or a plurality of samples from people who are not the subject. Any of the entries for which the sample SNP profile matches the subject SNP profile may be annotated as being from the subject. Preferably, the selecting step excludes, from the selected entries, the entries that were made through analysis of the one or more samples from the subject.

In some embodiments, a reported somatic tumor SV is confirmed by an orthogonal validation assay. For example, the identifying step may include includes short-read sequencing-by-synthesis of the tumor DNA; the orthogonal validation assay may include sequencing the tumor DNA using fluorescently labeled di-deoxy chain terminators to generate a chromatogram and operating software to make base calls from the chromatogram; and the somatic tumor SV may be confirmed to exclude artifacts in the results from the short-read sequencing-by-synthesis.

In certain embodiments, methods include designing and providing reagents useful to detect the somatic tumor SV in a sample from the subjection. The reagents may include PCR primers useful to specifically amplify the somatic tumor SV. Methods may include obtaining a bodily fluid sample from the subject after the subject has been treated to remove the tumor and conducting a digital PCR assay on the bodily fluid sample using the PCR primers to detect the somatic tumor SV present as circulating tumor DNA as a marker of minimal residual disease.

The identifying step of the methods may include sequencing the tumor DNA from the subject to obtain sequence reads, mapping the sequence reads to a reference, and determining that the reads map to the reference in a pattern indicative of the tumor SV. The tumor DNA may be obtained from a formalin fixed, paraffin embedded sample of the tumor. The subject SNP profile may be obtained from the sequences of the tumor DNA from the subject. The subject SNP profile and/or at least one of the sample SNP profiles are the results of a SNP genotyping assay that uses a bead array. In certain embodiments, the searching step includes comparing breakpoint genomic coordinates of the tumor SV with breakpoint genomic coordinates of the sample SVs.

### DETAILED DESCRIPTION

Methods of the invention provide filtering approaches to flag structural variants (SVs) as likely germline or somatic from NGS data without the need to sequence non-cancerous material (matched normal) from the same patient. In cancer genomics, somatic (cancerous) mutations and germline mutations have conventionally been distinguished by their presence or absence in sequencing data derived from healthy cells from the same patient, with mutations found exclusively in the tumor flagged as somatic. However, this approach has several limitations, both in terms of the added cost and complexity of processing and sequencing an additional sample from each patient, as well as the fact that a normal sample may not be available from all patients. Somatic mutation calling is hampered by artifacts that occur during NGS or by downstream bioinformatic approaches. While such artifacts are recurrent, they are not necessarily represented in every sample, and thus can be misclassified as somatic events.

Structural variants (SVs) are large-scale genomic alterations that involve re-arranging the genetic material of a cell through inversions, duplications, large-scale deletions, or inter-chromosomal translocations. In contrast to single nucleotide variants and small insertions and deletions, which affect precise genomic loci and can be found in multiple tumor/patient samples, SVs may be unique to a specific tumor due to the large-scale nature of the events and potential combinations of alterations. Thus, it is extremely unlikely that a somatic SV is identical between different tumors, and the associated SV breakpoints of each are considered unique. However, germline SVs may be present, and those may have identical breakpoint positions even among different people as those variants are passed down (effectively copied) from generation to generation.

Given that an SV observed in multiple tumor samples is very likely to be germline or a technical artifact, methods of the invention flag an SV as germline or somatic using only tumor sequence such as from whole-genome sequencing (WGS) and the resulting SV calls without the need for sequencing normal material from the same patient (“matched normal”). By storing in a database all SV calls and associated SNP profiles from all tumor samples analyzed previously, methods of the invention can compare the SV calls from a newly sequenced tumor sample against the previously identified SVs. Previous samples that with SNP profiles similar to the target sample are presumed to originate from the same patient and may be excluded from analysis. Of the remaining samples, any SVs which are observed in both the target and previously sequenced samples are flagged as germline/or artifacts and as such are filtered.

By generating and/or accessing a database of SVs previously identified, true somatic SVs (those only found in a tumor sample) may be selected, identified, or reported.

In preferred embodiments, methods of the invention use NGS data from a tumor sample, and generate structural variants calls from that sample. Methods may use Whole Genome Sequencing (WGS) to fully capture the complexity of the genome, but targeted panel enrichment approaches may also be employed. Certain embodiments use low-pass WGS (lpWGS), described in greater detail herein. Any bioinformatic method for calling an SV is permitted and certain embodiments make use of workflows for SV detection from sequence reads described in greater detail herein.

As a tumor sample is analyzed and SVs are detected, those SVs may be added to the database. The SVs added to the database may be understood to be “potential” somatic SVs. However, those potential SVs may include data artifacts, technical artifacts, germline SVs, and true somatic SVs. Data artifacts include, for example, bioinformatic mapping errors that may lead to an identical SV being called across multiple samples. Such data artifacts may occur with very short reads, reads from genomic regions of gene duplication or copy number variation, or other such issues. Technical artifacts may arise due to the nature of the sequencing approach, resulting in errors that generate spurious SV identification. One example of a technical artifact is template crossover between clusters when performing bridge amplification on a flow cell. Some potential SVs identified by methods herein include germline SVs, which generally include SVs that are shared across multiple individuals in a population and are not unique to the tumor. Some germline SVs may be benign or have no clinical significance. Methods herein use the database from other samples, from other people, to identify (and filter out) germline SVs. Further, methods herein may use orthogonal validation, such as by Sanger sequencing, to confirm putative somatic SVs. By those techniques, methods of the invention are useful to identify true somatic SVs, which include SVs that are only found in the patient tumor.

Preferred embodiments make us of a SNP profile from a given case. The SNP profile could be obtained using the same sequencing data or from a separate method (e.g. bead array).

The sample being analyzed, from a subject, is associated with a subject SNP profile and the prior samples represented in the database are each associated with their own sample SNP profile. Any suitable information or format may be used for a SNP profile such as text file listing SNP written with respect to a published human genome, e.g., hg38; Chr 9 1386A>T (or any other suitable format). The database also includes, for the prior samples, a list of any structural variants that were detected from previously sequenced samples (which may have been analyzed using the same approach as the target sample). The sequencing data in the database may be derived either from tumor or healthy material.

Methods make use of the sample SNP profile from previously sequenced samples. Those sample SNP profiles may be generated from either tumor or healthy material. The SNP profile could be obtained using the same sequencing data or from a separate method (e.g. bead array).

The SNP profile of the target sample is first compared to the SNP profile each previously sequenced sample, and the relatedness of each pairing is calculated. Any pairing with a relatedness above a given threshold is considered as originating from the same patient and removed from downstream analysis. Any suitable threshold may be used. For example, the threshold could be two SNP profiles that share a simple majority of their SNPs, or two profiles that have >95% similarity, etc.

In some embodiments, the breakpoint genomic coordinates of each SV in the target sample are compared against the genomic coordinates of all SVs detected in all previously sequenced samples. Any SV where both sets of breakpoint coordinates match with a previously detected sample may be removed from downstream analysis (e.g., as presumptively germline or benign). Matching could require an exact match or could permit a small window around each breakpoint is permitted to account for uncertainty in calling the precise position of a breakpoint. A suitable window would be +/−2 bp.

Various embodiments of methods herein may use additional or alternative features. For example, the size of the previously sequenced patient database could be capped (ex. 10,000 patients). As the human genome size is limited, and different regions of the human genome are mutated at different rates, it is possible (although unlikely) that a somatic SV with identical breakpoints could be observed. Setting the size of the database can ensure that the database represents a high number of germline SVs and artifacts.

The database could be enhanced by confirmation of the SVs by orthogonal validation to identify true somatic SVs and those that are germline. For example, in some embodiments, SVs are initially detected by subjecting formalin-fixed, paraffin embedded (FFPE) tumor slices to low-pass whole genome sequencing (LP-WGS) (discussed in greater detail below) using NGS to generate short reads that are input into a variant caller software package (also discussed in greater detail below). Any putative SV could be confirmed by an orthogonal validation assay such as, for example, by Sanger sequencing, i.e., sequencing the tumor DNA using fluorescently labeled di-deoxy chain terminators to generate a chromatogram and operating software to make base calls from the chromatogram.

The database could be further enriched using germline SVs identified in external databases (ex. gnomAD). Pathogenic variants or common breakpoints could be marked and not removed by this approach. i.e. SVs in specific regions would not be dynamically filtered out as breakpoints may be less unique.

Somatic SVs identified by methods herein may be used to report the presence/absence of an SV (i.e. as a comprehensive genomic profiling test) or used to design personalized assays for minimal residual disease (MRD). As discussed in greater detail herein, for certain approaches to an MRD assay, a bodily fluid sample, such as blood or plasma, is obtained, and cfDNA is extracted from the sample. Preferably up to 1.5. μg of cfDNA (e.g., a quantity between 100 ng and 1.5. μg) is extracted. By having analyzed tumor DNA from a known tumor sample, methods use primers that are designed to detect and amplify only tumor-specific variants, preferably structural variants (SVs) that are specific to the tumor. From the bodily fluid sample, a pre-enrichment may be performed, e.g., to specifically pre-amplify those tumor-specific variants, e.g., SVs.

The detection is specific for tumor nucleic acid by virtue of the use of primers designed by analyzing sequence data from tumor DNA obtained from a known tumor sample that has been obtained prior to tumor removal. The initial sequence data analysis, which may involve next-generation sequencing (NGS) of a tumor sample such as from a biopsy or a formalin-fixed, paraffin embedded (FFPE) tumor slice, and may proceed by low-pass, whole genome sequencing (LP-WGS), and may be performed at one point in time to detect a plurality of tumor-specific variants.

In various embodiments described herein, the sequence data may be obtained by sequencing DNA from an FFPE slice of the tumor; a library preparation protocol tailored to FFPE-sourced nucleic acid may be used; the tumor nucleic acid may be sequenced by LP-WGS; a computer system may be used to detect and rank tumor SVs and select a marker variant and to design primers specific for the marker variant; the primer pair may be used in a detection assay for a subject that has undergone treatment to eradicate the tumor; the sample may be a blood draw, or “liquid biopsy”, the detection assay may involve digital PCR with the sample in aqueous partitions using an amplification reaction and fluorescent probes; detecting fluorescence from the aqueous droplets may indicate the presence of the tumor nucleic acid in the sample; and/or the assay may be performed to detect minimal residual disease after the treatment.

### FFPE DNA Extraction

Methods of the disclosure may include obtaining nucleic acid from a formalin-fixed, paraffin embedded slice of a tumor so that the tumor nucleic acid may be sequenced. Tissue obtained by biopsy or surgery for pathological examination may be fixed in a fixative, such as formalin and embedded in paraffin, yielding formalin fixed, paraffin embedded (FFPE) blocks. Small (e.g., a few micrometer-thick) sections may be sliced from the blocks and stained on slides for microscopic analysis. Such slides are typically retained as a pathology archive.

Methods herein may use protocols for extracting DNA from FFPE samples and preparing high-quality sequencing libraries from the FFPE-extracted DNA. To extract nucleic acid, the sample is loaded into a tube such as microcentrifuge tube. A tissue lysis buffer and proteinase K (PK) solution mix may be added to the tube. Steps of protocols herein may be performed using reagents and material sold under the product name truXTRAC FFPE total NA (tNA) Ultra Kit by Covaris. The FFPE sample may be immersed in the tissue lysis buffer/PK solution mix and sonicated in a ultrasonication instrument according to manufacturer instructions for paraffin emulsification. The steps may be performed in laboratory test tubes, wells of a plate, microcentrifuge tubes, or tubes in a multi-tube strip.

After the tube is collect, it is centrifuged, e.g., spun at 5 k g for about 15 minutes, to form a pellet that includes DNA, preferably at least 100 ng, optionally even up to 1.5 μg. The described protocols provide high quality DNA, suitable or sequencing, with high yield (e.g., between 1 and 2 μg) from FFPE tissue samples. Preferably, the pellet is rehydrated with a suitable buffer such as buffer BE from Covaris and more preferably a tissue lysis buffer/PK solution mix is used. The tube may be sonicated to resuspend material of the pellet, and optionally treated with RNase. A DNA purification column may be placed into a collection tube. The sample is transferred into the column and the tube spun. Following DNA purification protocol instructions, the column is washed with buffer(s) such as BW Buffer and B5 Buffer (Covaris). Finally, the column is eluted with an elution buffer, eluting the DNA from the column. The collected (eluted) DNA preferably includes at least 100 ng, optionally about 1.5 μg, and may be analyzed or stored long-term. Methods of the disclosure produce high quality and high yield sequencing libraries from FFPE-extracted DNA.

### Library Preparation

Having extracted DNA from a sample, methods may include library preparation, which generally includes fragmentation, adaptor ligation, and amplification. When the source is a tumor biopsy, nucleic acids in very small quantities, or preserved (e.g., FFPE) sample, extracted DNA may be fragmented via a fragmentation step that may be more gentle and less damaging than conventional protocols. In some embodiments, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. In some embodiments, it may be preferable to produce fragments that have an average size with a peak approximately within the range of about 500, preferably at least about 600 or 700, and most preferably at least about 800 base pairs (bp) to 1,000 bp. A cocktail of restriction enzymes may be composed that will, on average, cut genomic DNA on about 800 to 1,000 base intervals. Preferred embodiments use a sonicator or adaptive acoustic focusing (AFA) instrument (Covaris). Embodiments may use a Qubit instrument to evaluate quantity and/or a TAPESTATION automatic electrophoresis instrument to evaluate fragment length, using manufacturer's literature for guidelines for the sonication instrument. One approach is to shear a very small sample to the desired optical density to establish the instrument settings to be used for the bulk of the sample. The resultant shearing protocol produces 800 to 1000 base fragments.

The fragments may be repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA repair enzymes and Structure-specific endonucleases are enzymes that cleave DNA at a specific DNA lesion or structure. Those enzymes can be used for repair of DNA from sample degradation due to oxidative damage, UV radiation, ionizing radiation, mechanical shearing, formalin fixation (post extraction) or long-term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

In some embodiments, the end-repaired fragments are purified using magnetic beads and a magnetic separation device. A bead to DNA fragment ratio of about 0.7× may be used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. One feature of embodiments of the disclosure may be a minimal or low-bead ratio, which, in combination with the fragment length and subsequent steps, provides high quality, high-yield sequencing libraries from FFPE samples. Enzymes or other reagents may be washed away, and DNA may be eluted into a ligation mix.

Methods may include ligating adaptors to the fragments to form adaptor-ligated fragments. Any suitable approach may be used. Some embodiments include dA tailing at the 3′ ends of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). The adaptor ligated fragments may be subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp.

The selected adaptor-ligated fragments may be amplified to obtain amplicons. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage. The result is a plurality of clonal amplicons copied from nucleic acid in a tumor sample. The amplicons may have sequencing adaptors or any suitable primer binding sites at either or both ends. At this stage, a library preparation is complete.

The described extraction and library preparation protocols may be optimized, compared to commercially available kits and protocols, to compensate for damage that is characteristic of FFPE samples and their extraction. For example, after emulsification of the paraffin, DNA may be subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of the steps—limited fragmentation, gentle bead clean-up, and purification after adaptor ligation with size-selection step—may contribute importantly to the preparation of high-quality sequencing libraries from FFPE samples.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline SVs. Such an analysis may provide an identification of tumor-specific somatic SVs that are present in a patient's tumor DNA. That information is then used to design reagents to assay future samples from the patient for those same tumor-specific somatic SVs. For example, an informatics pipeline may be used to design amplification primers and fluorescent probes for the detection of such variants by a digital PCR assay. Some embodiments identify tumor-specific SVs present in a patient's tumor DNA and then use an informatics pipeline to design primers and fluorescent probes useful for detecting by digital PCR those SVs in cell-free tumor DNA in blood or plasma, e.g., from a liquid biopsy.

### Sequencing

Nucleic acid obtained according to methods of the disclosure is preferably sequenced to obtain sequence data. For example, methods may include sequencing DNA from a tumor sample from the subject to obtain sequence reads.

Sequencing may be by any method known in the art. Suitable DNA sequencing techniques may include the dideoxy chain-termination sequencing technique known in the art as Sanger sequencing, which uses labeled terminators and gel separation in a slab or capillary. Sequencing may include the sequencing by synthesis using reversibly terminated nucleotides and the detection of pyrophosphate in the technique known as pyrosequencing commercialized by ROCHE 454. Sequencing may proceed by techniques that include allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes. Sequencing may be performed using one of the single molecule, long read sequencing platforms commercialized by HELICOS, PACIFIC BIOSCIENCES, or OXFORD NANOPORE.

Sequencing techniques and instruments that may be used include, for example, those offered by ILLUMINA, INC. or ULTIMA GENOMICS. Illumina sequencing is based on the amplification of a sequencing library described above on a solid surface of a flow cell using fold-back PCR and anchored primers. Amplicons of adaptor-ligated fragments that constitute the sequencing library are annealed to oligos attached to the surface of flow cell channels that are extended by which the amplicons are bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing generates sequence data and for short-read, ensemble sequencing platforms such as the ILLUMINA platform, the sequence data comprises a large number of short sequencing reads typically accessible from the ILLUMINA system in a computer file format known as FASTQ.

The sequencing instrument and technique relates to the biochemistry of base determination and also implicates read length and read number, with consequences for read assembly. For example, the output from Sanger sequencing on a glass-capillary instrument provided by ABI is typically a small number of medium length (several hundred bases) chromatograms that are provisionally “called” (interpreted) as bases by software and presented visually for human verification. Long read sequencing (e.g., PACIFIC BIOSCIENCES, OXFORD NANOPORE) is meant to provide single or low numbers of much longer (>1,000) base reads. Short read sequencing (e.g., ILLUMINA) provides a large number (e.g., millions) of short reads (e.g., 50 or fewer bases) that are typically mapped to a reference and/or assembled de novo to show the original sequence. Illumina is accepted as an industry standard example of a next-generation sequencing (NGS) platform. Whatever instrument or technique is used, methods may include one or any combination of suitable “coverage” strategies, which involve determinations of what targets to sequence and at what coverage.

Coverage strategies may include, for example, transcriptome sequencing in which all RNA transcripts are sequenced redundantly, re-sequencing in which a presumptively very similar genome is known and only highly variable targets are sequenced, whole exome sequencing in which all expressed genes or exons are sequenced, or other coverage strategies. Even with a particular coverage strategy, one may opt for a certain depth of coverage. For example, for some applications, when NGS is used, 30× coverage is considered a standard coverage in which substantially all bases are sequenced redundantly such that each base, on average, appears in about 30 unique sequence reads. Certain preferred embodiments of the invention use low-pass whole genome sequencing (as used herein, “whole genome sequencing” means that a substantial portion such as at least 70% or 90% of a genome or at least the equivalent of at least one chromosome is sequenced). Low-pass whole genome sequencing (LP-WGS) is a technique in which each base is sequenced a few times (known as low-depth coverage) e.g., with a depth of coverage below about 15, even 5, and as low as 0.1-1 times. By reducing the depth of coverage, the cost of sequencing the whole genome is reduced while maintaining genome-scale coverage. LP-WGS is described in Christodoulou, 2023, Combined low-pass whole genome and targeted sequencing in liquid biopsies for pediatric solid tumors, NPJ Precision Onc 7:21 and Zheng, 2022, Experience of low-pass whole genome sequencing-based copy number variant analysis, Diagnostics (Basel) 12(5):1098, both incorporated by reference.

Whatever technique and coverage are employed, methods include sequencing nucleic acid from a tumor. In certain preferred embodiments, LP-WGS is used to sequence substantially at least about 60 or 70 or 80 or 90% of a tumor genome at a coverage of about 15× or lower (e.g., 5×). The sequencing provides sequence data of the tumor nucleic acids. The sequence data may be analyzed to create a personalized tumor mutation profile, which includes any potential tumor variants and/or mutations. Low-pass sequencing is especially well suited for methods described herein where an objective is to detect SVs because, compared to other genotyping purposes, SVs are readily discoverable by LP-WGS as described herein.

A variety of different variants and mutations may be tracked using the tumor mutation profile. Typically, these variants are structural variants. Structural variants (SVs) are genomic abnormalities that may amplify, delete, or rearrange genomic regions of a tumor. It is possible and, in fact, common for more than one SV to occur in the same tumor. As used herein, an SV generally refers to a rearrangement, duplication, or deletion of a segment of length of at least about 1,000 bases. Methods of the disclosure may also be used to detect tumor-specific polymorphisms and/or small indels.

### Detection of Tumor-specific Variants

The disclosure includes methods for analyzing sequence reads, as may be obtained from nucleic acid from tumors, to identify structural variants (SVs), and optionally filter out any putative structural variants that are not somatic (e.g., germline SVs or artifacts from sample processing or sequencing) to identify SVs that are specific to the tumor, i.e., tumor variants. Methods may include comparing tumor sequence to a reference by one or more algorithms, identifying structural variants in the tumor nucleic acid, and designing primers to specifically amplify those tumor variants. Sequence reads from tumor nucleic acid may first be cleaned up, mapped to a reference, and or subject to computational workflows to detect SVs.

Reads can be cleaned using known software methods such as fastp as described in Chen, et al., 2018, fastp: an ultra-fast all-in-one FASTQ preprocessor, Bioinformatics, 34(17):i884-i890, incorporated by reference. Cleaning may include trimming adapter sequences, removing low quality bases at the ends of reads and artifacts such as polyG tails. In some FFPE embodiments cleaning may include removing reads shorter than 30 bp instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated by reference. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, each incorporated by reference. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9):1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

A workflow for SV detection from sequence reads and for primer design may be automated using tools such as Snakemake or Nextflow and custom programming using R or Python, for example, to link input/output across the various workflow steps. Some embodiments employ a computational pipeline that uses two or more different algorithms, each intended for finding SVs, to call putative SVs and merge the results. The computational pipeline may be used for mapping reads to a reference by a first algorithm (in a first mapping) and also by a second algorithm to identify SVs by each algorithm and then selecting the better result or merging the results of the multiple mapping steps to describe the structural variants. One of the algorithms may be a graph-based algorithm. In preferred embodiments, the first algorithm adds the reads to a genomic graph and finds a path through the graph best supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27(12):2050-2060 and Cameron, 2021, GRIDSS2: comprehensive characterization of somatic structural variation using single breakend variants structural variant phasing, Genome Biol 22(1):202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read pairs to a reference and searches for genomic regions in the reference where a significant number of read pairs align to the reference in positions inconsistent with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9):677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7(8):1034-1047, incorporated herein by reference in its entirety. SplitSeq can be used to reconstruct the exact fusion sequence based on split reads and read pairs with one unmapped mate. Discordant reads can be re-aligned to reduce false positive SV calls. After merging of the SV calling paths using the first and second algorithms, the putative SVs can be annotated with genes that overlap SV breakpoints,

Methods may include filtering SVs that were identified by the mapping workflows to remove germline SVs and/or sample handling artefacts, thereby providing a set of somatic SVs, or tumor variants, present in the tumor DNA.

Methods of the invention make use of a database of genetic variants from previous samples. For each sample, the database includes SVs that were found in that sample as well as a record of single nucleotide polymorphisms, a sample SNP profile, associated with that sample. The tumor sample is from a subject and the database may include previous samples from that subject and other people. To analyze the tumor sample, methods include using a subject SNP profile—a record of single nucleotide polymorphisms present in the subject—to compare to sample SNP profiles in the database. A sample in the database with a sample SNP profile that matches the subject SNP profile is presumed to be from the same person as the subject, albeit from previous visit or analysis. Database samples from the same person are excluded from further analysis, and the SVs from the tumor sample are compared to SVs in the non-excluded database records, which are from people other than the subject. Where SVs from the tumor sample match SVs found in samples from other people, the tumor SVs are called as being germline. When the tumor SVs do not have any matches among data from other people, those SVs are called as somatic.

Methods may optionally further involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, for an MRD assay to achieve excellent sensitivity and specificity, all SVs found by sequencing are preferably filtered to remove benign germline variants from the putative set, leaving a set of tumor-specific somatic SVs. Filtering may include comparing to a database of known SVs to remove from consideration those that are documented to be benign. Such a database may include the Genome Aggregation Database (gnomAD) described in Chen, 2023, A genomic mutational constraint map using variation in 76,156 human genomes, Nature 625:92-100, incorporated by reference; Genome in a Bottle SVs described in Chapman, et al., 2020, A crowdsourced set of curated structural variants for the human genome, PLOS Comp Bio, 16(6): e1007933, incorporated by reference; or the database of human structural variation known as dbVar described in Lappalainen, 2013, DbVar and DGVa: public archives for genomic structural variation, Nucleic Acids Res 41(Databse Issue):D936-41, incorporated by reference.

The described workflows provide for mapping the sequence reads to a reference and identifying read mappings that indicate a structural variant in the tumor nucleic acid, relative to the non-tumor nucleic acid of the subject. That structural variant is tumor specific. It is a variant specific to the tumor, herein referred to as a tumor variant. Using methods of the disclosure, the tumor variant is found by sequencing tumor nucleic acid and analyzing the sequence data. A feature of the disclosure is that such a tumor variant may be confirmed by orthogonal testing. Thus, the invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### Orthogonal Validation

The disclosed methods are used to detect and report one or more tumor-specific variants that constitute a tumor signature. Following identification of the tumor-specific variants, methods may include performing additional analysis to validate that any such variant is, in fact, present in the tumor genome (to remove false positives) and also, in fact, not present in healthy, non-tumor DNA (correct for false negatives).

Preferably, the method used for confirmatory testing of a tumor variant in a patient is orthogonal to the method used to create the tumor mutation profile, or signature. The use of this orthogonal testing method offers a distinct error profile from the method to identify the candidate SVs. That is, where a tumor variant is detected by NGS techniques, the variant is validated by some separate, other technique. While either technique may by itself have some bias or error, the use of an orthogonal technique confirms the variant but only with different, dissimilar potential sources of bias or error. There is a very low probability that an error introduced by a primary detection technique will be exactly mimicked by an orthogonal detection technique. Thus, consensus between the two detection techniques is strong indication that the detected tumor variant is a true tumor variant. Use of an orthogonal testing method mitigates the risk of false positives and/or negatives. In certain embodiments, the orthogonal testing method comprises testing the detected tumor variant that was sequenced against normal tissue to test for presence of clonal hematopoiesis of indeterminate potential (CHIP) or Germline SVs. The orthogonal testing method may also be performed against an unmatched control (i.e., DNA not derived from the patient) to detect the presence of spurious amplification or false positives. The orthogonal testing method can test for the presence of clonal hematopoiesis of indeterminate potential (CHIP) or Germline SVs as well as for SVs present in the tumor. The orthogonal testing method may also be used to accurately determine specific characteristics of a variant, e.g., the copy number of a SV.

The orthogonal test may be performed by a variety of different methods. Various testing methods may be chosen depending on the specific needs of the patient, the availability of the test to the physician, or various other factors. Any orthogonal test may be used as long as it carries a separate error profile to the initial methods of the previous step. Preferred embodiments use NGS as a primary variant detection technique to detect a variant and use a suitable orthogonal detection technique to confirm the detected variant.

Any suitable test may be used for the orthogonal detection technique including, for example, quantitative PCR (qPCR), ELISA, restriction fragment length polymorphism (RFLP) and similar, optical mapping, reverse transcription polymerase chain reaction (RT-PCR), transcription-mediated amplification (TMA), ligase chain reaction (LCR), targeted re-sequencing, RNA sequencing, Sanger sequencing, single molecule sequencing, atomic force microscopy sequencing, fluorescent in situ hybridization (FISH), a fluorescent-probe based DNA microarray, in vitro transcription, nanopore sequencing, long read sequencing and protein sequencing or detection, others, or combinations thereof.

In some embodiments, the orthogonal detection technique includes Sanger sequencing. Sanger sequencing does not require the same combination of PCR and bridge amplification steps that are involved in NGS and the output of Sanger sequencing is chromatograms that are much longer than short-reads e.g., from NGS. Due to the output length, Sanger sequencing is much less prone to assembly errors for short copy number variations such as dinucleotide and trinucleotide repeats and also less prone to base calling errors by software in the presence of small indels. Sanger sequencing instruments have matured over decades of use and refinement and use electrophoretic gel separation in glass capillaries of fluorescently labeled fragments. Sanger sequencing is good enough that it is accepted in the primary literature as the “gold standard” against which any new sequence analysis technique must be proven. There is one known issue of a mis-labeled fragment being driven through the capillary past the detector simultaneously with correctly-labeled fragments. But that issue is complementary to the types of problems that NGS has with amplification bias and homopolymer runs. For those reasons, Sanger sequencing offers an excellent orthogonal validation technique for use in methods of the disclosure.

In certain embodiments, the orthogonal detection technique includes atomic force microscopy (AFM). AFM uses a cantilever tip that can interact with DNA where input force required for motion of a base is characteristic allowing base sequence to be read from the instrument run. AFM is sensitive to secondary structures, bound proteins, and packaging. AFM is potentially useful to scan DNA in situ, e.g., within a fixed sample without liberation into solution. As tumor variants will disrupt assembly of the transcription complex and knock out binding sites for DNA binding proteins, AFM can show the bound proteins—or lack of bound proteins—and absence of complexes and structure that would only be consistent with the tumor variants identified by NGS.

The orthogonal detection may optionally include restriction fragment length polymorphism (RFLP) and similar techniques (such as amplified fragment length polymorphism (AFLP) and/or optical mapping. Techniques such as RFLP, AFLP, and optical mapping all give a characteristic and very specific output based on where, or in what pattern, restriction enzyme cut DNA. A tumor SV will change a restriction pattern, relative to non-tumor DNA, in two significant ways. As “SV” stands for structural variant, the tumor DNA differs from non-tumor DNA because large (>1 kb) segments have been moved, removed, duplicated, or inverted. This will create a very different restriction pattern. Additionally, tumors are hyperproliferative and tend to have extensive hypomethylation of genes and promoters where, for example, methylation of promoter of an oncogene in healthy, non-tumor DNA inhibits expression of the oncogene, a common phenomenon in cancer is hypomethylation and associated expression of the oncogene. Classes of restriction enzymes are methylation specific and such methylation-specific restriction enzymes may be used once a tumor SV is identified by NGS to validate that restriction patterns that would be expected in the tumor DNA are observed in the tumor DNA.

In some embodiments, the orthogonal detection technique includes FISH or a fluorescent microarray. Both are examples of techniques in which sample DNA is exposed to sequence-specific probes that anneal to the sample DNA when and only when a target of interest is present. Once a tumor SV is identified by NGS, a plurality of probes (e.g., dozens, hundreds, or thousands, etc.) in which some number can be designed to hybridize only to nucleic acid that includes the tumor SV; some hybridize only to nucleic acid that does NOT include the tumor SV, some hybridize to both tumor and non-tumor nucleic acid, and some may hybridize only to some extrinsic control nucleic acid. Tumor nucleic acid and non-tumor nucleic acid are exposed to the probe set and patterns of hybridization are read (e.g., fluorescently) to validate that a pattern that would be expected (if the SV is a true tumor-specific SV) is observed in the assay data. FISH may be preferred where the original sample is, for example, an FFPE slice of the tumor on a slide, as the FISH probes may be used to interrogate the slice in situ on the slide.

Because the detected variant is validated by an orthogonal detection technique that is unlike the primary detection technique, any bias or systematic error in either technique is corrected for by the inclusion of the other.

Having validated the identity of the truly tumor-specific variant, the tumor SV may be used in an assay for the presence of the tumor, particularly in an assay for minimal residual disease after treatment for the tumor. In particular, a primer pair may be designed that amplifies the SV or that flanks a breakpoint of the SV and amplifies a segment containing the breakpoint. When a patient is treated to eradicate the tumor, the primer pair can subsequently be used to detect whether any tumor nucleic acid is still present in a sample from the patient.

### Selecting a Set of Marker Variants

Methods of the disclosure include a step of selecting from any and all detected tumor SVs a subset of those that constitute a set of marker variants to be used in a digital PCR MRD assay. Criteria for selecting the marker variants may include level of duplication; driver mutations; passenger mutations; truncal mutation; conservation across tumor clonality; suitability for PCR; other criteria; and any combination of the foregoing. The invention includes methods for ranking structural variants (SVs) and/or otherwise detecting and assigning relative ranks, in terms of selection for an MRD assay. Systematically ranking SVs provides an approach for the automatic selection of which SVs to interrogate in a diagnostic assay, such as a digital PCR assay for MRD from circulating-tumor DNA in blood or plasma. Methods include analyzing sequence data from tumor nucleic acid from a tumor of a subject to identify the presence and copy numbers of a plurality of tumor-specific structural variants (SVs) in the tumor nucleic acid compared to non-tumor nucleic acid from the subject; ranking the SVs wherein higher ranks are correlated to higher copy numbers; and providing reagents for an assay that detects a tumor signature comprising one or more of the SVs selected for having the higher ranks. Thus, methods may include determining copy number of detected SVs. Copy-number calling can then be performed to, for example, estimate tumor cell content in the sample and the degree to which the tumor genome may be rearranged. Genome-wide copy number information can be used later for prioritizing SVs for validation. Exemplary copy-number analysis can include ichorCNA described in Adalsteinsson, et al., 2017, Scalable whole-exome sequencing of cell-free DNA reveals high concordance with metastatic tumors, Nature Comm volume 8:1324, incorporated by reference.

Other criteria may also be used for selecting marker variants. For example, ranking may also include assigning a high rank to a truncal SV identified as an initiating truncal mutation of the tumor. The ranking step may include application of any other suitable criteria, such as the requirement for suitable primer binding sites by which one primer pair could amplify the multiple loci or instances of duplication of the marker variant. In certain tumor signature embodiments, the computer system is used to design multiple primer pairs that are useful to detect two or more of the SVs with higher ranks as a patient-specific, tumor-specific signature of the tumor in the subject.

### Assay Design

Methods may include designing and providing a plurality of copies of a primer pair that specifically amplify the sequence and storing the plurality of copies of a primer pair as reagents for use in one or more future assays for minimal residual disease. Designing the primer pair(s) may be implemented by a computer system. The computer system may also be used to design any or all other aspects of detection assay for the marker variant(s). The computer system may be used to design, for example, suitable fluorescent hydrolysis probes such as the probes sold under the trademark TAQMAN by Thermo Fisher Scientific (Waltham, MA). The computer system may be programmed to calculate and store conditions for a digital PCR (dPCR) assay such as: sample volume, dilution factor, partition number, reagent concentration, instrument settings, excitation and detection wavelengths, others, or any combination thereof. Output parameters from the computer system may be used as inputs to a suitable dPCR instrument or system, optionally carried from the assay design pipeline to the dPCR system by a direct data connection (e.g., WiFi or LAN) or by a managed system such as a laboratory information management system (LIMS).

From that, the computer system may thus provide conditions for an amplification reaction that will use a plurality of primer pairs designed to amplify a respective plurality of structural variants (SVs). The computer system output may also specific information such as sequences for the primer pairs that will amplify copies of the one or more SVs. Oligonucleotide reagents (primer pairs and fluorescent probes) maybe synthesized or obtained from a vendor such as Integrated DNA Technologies (Corralville, IA) and transferred (i.e., pipetted or dispensed) into reservoirs of the dPCR system. For example, the plurality of primer pairs are obtained as a reagent in one or more containers such as reagent tubes that are provisionally stored (e.g., lyophilized or in a freezer) and separately, subsequently dispensed to the detection assay instrument for use in the amplification reaction for detection of the plurality of SVs as a tumor-specific, patient specific signature of presences of the tumor.

### Detection Assay

Methods herein may include performing an assay to detect a marker variant in a sample from a subject and reporting the presence of the tumor in the subject when the assay is positive for the marker variant in the sample. The DNA used as input into the MRD detection assay may include cfDNA extract from a bodily fluid sample. The input DNA may include copies of cfDNA made by an amplification or pre-amplification. The input DNA may include both cfDNA extracted from a sample and copies made from among the cfDNA by a pre-amplification step. The pre-amplification step may be a linear amplification or PCR and may use primers specific to tumor sequences (e.g., ctDNA) and thus enrich for ctDNA among cfDNA.

The disclosed methods are useful for detection of any suitable target of interest in a sample. For example, methods are useful to detect nucleic acid from a pathogen in a mixed environmental sample or in a clinical sample that includes abundant host nucleic acid. The method may be used to detect fetal DNA in maternal blood or plasma. In certain preferred embodiments, the method is useful to detect a variant associated with a disease, such as an SV from tumor DNA present among cell-free DNA in a sample from a patient.

In preferred liquid biopsy and dPCR for MRD embodiments, obtaining a sample may involve receiving one or more blood collection tubes or containers containing blood or plasma that was obtained from the subject via blood draw. The sample may include cell-free DNA from blood or plasma from the subject.

In certain optional in vitro “pre amplification” embodiment, the detection may proceed by at least two distinct stages or mechanisms that include (i) copying the marker variant using variant-specific primers and tailed primers to form tailed amplicons and (ii) amplifying the tailed amplicons in the presence of probes that indicate the presence of amplicons from the target of interest in the aqueous compartment. In some embodiments, a pre-amplification step may use primers designed to specifically amplify the marker variant. For example, if the variant is a structural variant, the pre-amplification may use a pair of primers designed to anneal to nucleic acid at locations that flank a breakpoint of the structural variant. This strategy further enriches the sample for copies of the marker variant, ensuring that the presence of the variant is detected in the subsequent detection steps. This pre-amplification step specifically addresses problems associated with some dead volume of sample that resists detection by existing digital PCR (dPCR) approaches. Due to the stochastic nature of sampling, some very minor fraction of a sample might otherwise, by chance, typically go undetected by dPCR. Here, the pre-amplification step may increase quantity of the marker variant prior to the partitioning and dPCR detection, reducing the likelihood that the target of interest will be undetected due to stochastic loss in dead volume. After the pre-amplification, the sample may be partitioned into aqueous compartments.

Regardless of any optional in vitro “pre-amplification” embodiments, the detection assay is useful to detect the tumor, and may be used as an MRD assay using at least 100 ng of DNA as input, preferably at least about 1 microgram and even, e.g., up to about 1.5 microgram as input.

### Digital PCR

The described detection assay may be any suitable assay including, for example, nucleic acid sequencing, DNA microarray analysis, fluorescent in situ hybridization, PCR, quantitative PCR, or digital PCR (dPCR). In preferred embodiments, the detection assay is dPCR and the sample comprises blood or plasma from the subject and the assay uses at least 100 ng cfDNA (and/or copies made from cfDNA) as input. For the assay, dPCR may include partitioning at least a portion of the at least 100 ng into aqueous partitions that include PCR reagents and fluorescent probes for the amplicons and conducting the amplification reaction in the aqueous partitions. Where the input comprises 1.5 μg and steps are performed to protect against saturation, a portion of the 1.5 μg, e.g., 750 ng may be used as input into the dPCR. If the dPCR result is saturated (all or substantially all partitions blank), a second portion of the 1.5 μg may be taken, diluted, and partitioned for a second round of dPCR. The assay comprises performing an amplification reaction to detect amplification of the copies of the one or more SVs.

The assay includes partitioning the sample into aqueous partitions and performing an amplification reaction in the aqueous partitions using at least one primer specific for the sequence and a probe that provides a signal when the amplification reaction using at least one primer generates an amplification product. The method may include partitioning the sample into aqueous partitions that include PCR reagents and fluorescent probes for the amplicons, conducting the amplification reaction in the aqueous partitions, and detecting fluorescence from the partitions to detect the residual presence of the tumor after the treatment. Those dPCR steps may all be automated and/or performed using a commercially available dPCR instrument or system. The dPCR system may detect fluorescence from the partitions and provide output indicating a number of partitions that include the marker variant.

For a subject in whom a tumor has been diagnosed, and a sample of the tumor (biopsy, FFPE slice) obtained, a treatment may have been administered to eradicate the tumor. For example, the person may have undergone surgical resection to remove the tumor, radiation therapy to ablate the tumor, or chemotherapy to kill cells of the tumor. The person may spend some amount of time feeling the benefit of the treatment, living a cancer-free life. However one issue with cancer treatment is that, after treatment to eradicate a tumor, non-tumor cells in the person may shed a super-abundant quantity of cell-free DNA that is not tumor DNA into circulation, a phenomenon known as cfDNA leakage. For the few days immediately after tumor resection/treatment, cfDNA leakage may mask the presence of ctDNA and inhibit the detection of MRD. In such situations, the invention provides methods that provide a detection assay that can detect MRD even in the presence of overwhelmingly abundant non-tumor cell-free DNA. Thus, an MRD assay of the invention may be beneficial in the first few hour to few days to ten days after treatment to remove a tumor, or when a person has suffered other, incidental tissue injury, among elderly people or a patient suffering arthritis or any other condition associated with inflammation. By using at least 100 ng and even up to 1.5 μg of DNA as input into the MRD assay, the assay design avoids false negatives, has very high sensitivity, and detects the presence even of tumor DNA even when present as only a very minor fraction of cfDNA.

By the foregoing disclosure, methods are provided for detecting SVs from sequencing data from a tumor sample and classifying those SVs as somatic or germline without the use of matched normal sequences. Methods use a database of SVs and SNP profiles from previous samples. Methods include comparing a subject SNP profile to sample SNP profiles in the database and excluding from further analysis database entries with matching SNP profiles (as presumably originating from the same subject). Where SVs from the tumor sample match SVs found in samples from other people, the tumor SVs are called as being germline. When the tumor SVs do not have any matches among data from other people, those SVs are called as somatic.

What is claimed is

1. A method of detecting a tumor-specific mutation, the method comprising:
identifying (i) a tumor structural variant (SV) in sequences of tumor DNA from a subject, and (ii) a subject SNP profile describing single nucleotide polymorphisms in nucleic acid of the subject;
comparing the subject SNP profile to a database in which entries include sample SVs and a sample SNP profile from previous samples;
selecting, from the database, a set of selected entries for which the sample SNP profile and the subject SNP profile are dissimilar according to a test;
searching the selected entries for the tumor SV; and
reporting the tumor SV as germline when the tumor SV matches at least one of the sample SVs in the selected entries.

2. The method of claim 1, further comprising reporting the tumor SV as somatic when the tumor SV does not find a match among the selected entries.

3. The method of claim 2, further comprising confirm a reported somatic tumor SV by an orthogonal validation assay.

4. The method of claim 3, wherein the identifying step includes short-read sequencing-by-synthesis of the tumor DNA; wherein the orthogonal validation assay includes sequencing the tumor DNA using fluorescently labeled di-deoxy chain terminators to generate a chromatogram and operating software to make base calls from the chromatogram; and wherein the somatic tumor SV is confirmed to exclude artifacts in the results from the short-read sequencing-by-synthesis.

5. The method of claim 2, further comprising designing and providing reagents useful to detect the somatic tumor SV in a sample from the subjection.

6. The method of claim 5, wherein the reagents include PCR primers useful to specifically amplify the somatic tumor SV.

7. The method of claim 6, further comprising obtaining a bodily fluid sample from the subject after the subject has been treated to remove the tumor and conducting a digital PCR assay on the bodily fluid sample using the PCR primers to detect the somatic tumor SV present as circulating tumor DNA as a marker of minimal residual disease.

8. The method of claim 1, wherein the identifying step comprises sequencing the tumor DNA from the subject to obtain sequence reads, mapping the sequence reads to a reference, and determining that the reads map to the reference in a pattern indicative of the tumor SV.

9. The method of claim 8, further comprising obtaining the tumor DNA from a formalin fixed, paraffin embedded sample of the tumor.

10. The method of claim 1, further comprising obtaining the subject SNP profile from the sequences of the tumor DNA from the subject.

11. The method of claim 1, further comprising annotating any of the entries for which the sample SNP profile matches the subject SNP profile as being from the subject.

12. The method of claim 1, wherein the entries in the database were made by analyzing the previous samples, wherein the previous samples include one or more samples from the subject and a plurality of samples from people who are not the subject.

13. The method of claim 12, wherein the selecting step excludes, from the selected entries, the entries that were made through analysis of the one or more samples from the subject.

14. The method of claim 1, wherein the subject SNP profile and/or at least one of the sample SNP profiles are the results of a SNP genotyping assay that uses a bead array.

15. The method of claim 1, wherein the searching step includes comparing breakpoint genomic coordinates of the tumor SV with breakpoint genomic coordinates of the sample SVs.
